(19) United States
(12) Patent Application Publication
(10) Pub. No.: US 2018/0310860 A1
(43) Pub. Date: Nov. 1, 2018

(54) DISTANCE MEASUREMENT DEVICE AND IMAGING SYSTEM

(71) Applicant: SONY CORPORATION, TOKYO (JP)

(72) Inventor: KOICHIRO KISHIMA, KANAGAWA (JP)

(21) Appl. No.: 15/764,443

(22) PCT Filed: Sep. 5, 2016
(86) PCT No.: PCT/JP2016/076041

§ 371 Date: Mar. 29, 2018

(30) Foreign Application Priority Data
Oct. 19, 2015 (JP) 2015-205445

(51) Int. Cl. A61B 5/107; A61B 5/00; A61B 1/06; A61B 1/00; A61B 1/04; A61B 1/317; G01C 3/08; G01S 17/08

(52) U.S. Cl. CPC A61B 5/1079; A61B 5/0084; A61B 5/1076; A61B 1/063; A61B 1/00009; A61B 2505/05; A61B 1/00057; A61B 1/317; G01C 3/08; G01S 17/08; A61B 1/04

(57) ABSTRACT

A distance measurement device according to the present disclosure includes: a light source configured to emit pulsed laser light; a superimposition portion configured to superimpose reflection light obtained by reflection of the pulsed laser light by an object to be measured and reference light that is the pulsed laser light; a saturation output portion, on which the reference light and the reflection light superimposed on each other are made incident, configured to output light having a saturated light quantity when incident light reaches a predetermined light quantity due to superimposition of pulses of the reflection light and the reference light; and a light-receiving portion configured to receive the light outputted from the saturation output portion.

### TECHNICAL FIELD

The present disclosure relates to a distance measurement device and an imaging system.

### BACKGROUND ART

The endoscopic surgery requires a measurement of a thickness and a measurement of an area. For example, in the orthopedic surgery field, there is a need for measuring a thickness of a cartilage and the like. However, it is difficult to take an X-ray image of the cartilage and the measurement of the cartilage is usually performed by MRI with relatively low measurement accuracy. Also, an arthroscope is used as a method of magnifying and observing the cartilage. In addition, since MRI is expensive, many medical facilities are not equipped with MRI. As a result, the arthroscope is a main medical instrument for observing the cartilage. As such, in the orthopedic surgery field, there is a need for measuring a distance to an object that is observed by the arthroscope or the like.

On the other hand, a distance measuring technology has been used in various fields in recent years. As an example, a distance measuring technology is used for the purpose of preventing collision of automobiles or the like. This method uses a technology in which a distance is determined by radiating light from a light source (LED, LD, or the like.) to an object to be measured and obtaining a phase difference between radiated light and reflection light from the object to be measured. For example, Non-Patent Literature 1 below describes a distance measuring technology using a Time-of-Flight (TOF) method.

### CITATION LIST

### Non-Patent Literature

Non-Patent Literature 1: “Precise pulsed time-of-flight laser range finder for industrial distance measurements” edited by Ari Kilpela, Riku Pennala, and Juha Kostamovaara. Review of Scientific Instruments, Volume 72, Number 4, published in April 2001.

### DISCLOSURE OF INVENTION

### Technical Problem

However, in the distance measuring technology used for the purpose of preventing collision of automobiles or the like, for example, pulsed light having a frequency of 1 MHz is radiated from a light source and a phase difference (a phase time) between the pulsed light and reflection light is measured. Light travels through the air at about 300 [m] per 1 [μs], thus, if light is received using an element having a band (a time resolution) of 4 [GHz], a distance can be measured in a range of 0 [m] to 150 [m] with a resolution of 0.075 [m]. Further, it is currently possible to make a measurement with a time resolution of about 10 [GHz], thus this method allows a measurement with accuracy of about 0.03 [m] (3 [cm]). However, a resolution of 3 [cm] is not sufficient for application in the medical field, for example, for fulfilling a need, or the like for measuring the cartilage using the arthroscope in the orthopedic surgery field. Thus, a method of making a measurement with accuracy of 1 [mm] or less, or the like under a small-diameter endoscope, such as the arthroscope, frequently causing image distortion has not been available to date.

Specifically, the arthroscope or the like generally includes 2 kinds of optical paths for a lighting system and for an image transmitting system. A distance may be measured by introducing structured illumination into the lighting system and obtaining an illumination pattern. However, the arthroscope, which needs to be inserted into a joint, generally has a small diameter, thus an image observed by the arthroscope is distorted. An image of a flat test chart photographed by the arthroscope also demonstrates that the observation image is distorted. It is difficult to make an accurate distance measurement from such a distorted image even with a distance measuring means (trigonometry) using a usual optical camera or the like.

Further, the lighting system is configured to emit random divergent light without having an imaging lens, thus failing to form a pattern on a specimen. As a result, it is difficult to measure a distance with such a system. Further, in the image transmitting system, a straight line scanned by an imaging element becomes a curved line on the specimen due to curvature aberration or the like of the arthroscope. Such a curved line is however recognized as a straight line on the imaging element, making it further difficult to measure a distance.

Therefore, there has been a demand for accurately measuring a distance to an object to be measured in the medical field in which the endoscope such as the arthroscope is used.

### Solution to Problem

According to the present disclosure, there is provided a distance measurement device including: a light source configured to emit pulsed laser light; a superimposition portion configured to superimpose reflection light obtained by reflection of the pulsed laser light by an object to be measured and reference light that is the pulsed laser light; a saturation output portion, on which the reference light and the reflection light superimposed on each other are made incident, configured to output light having a saturated light quantity when incident light reaches a predetermined light quantity due to superimposition of pulses of the reflection light and the reference light; and a light-receiving portion configured to receive the light outputted from the saturation output portion.

In addition, according to the present disclosure, there is provided an imaging system including: a distance measuring unit including a light source configured to emit pulsed laser light, a superimposition portion configured to superimpose reflection light obtained by reflection of the pulsed laser light by an object to be measured and reference light that is the pulsed laser light, a saturation output portion, on which the reference light and the reflection light superimposed on each other are made incident, configured to output light having a saturated light quantity when incident light reaches a predetermined light quantity due to superimposition of pulses of the reflection light and the reference light, and a light-receiving portion configured to receive the light outputted from the saturation output portion; and an endoscope unit including an endoscope configured to emit the pulsed laser light made incident in the endoscope to the object to be measured, an imaging element configured to image the object to be measured as an object by the endoscope, and an adjustment portion configured to adjust a direction of the pulsed laser light such that the pulsed laser light is radiated to a specified location of the object to be measured.

### Advantageous Effects of Invention

As described above, according to the present disclosure, it becomes possible to accurately measure a distance to an object to be measured in the medical field in which the endoscope such as the arthroscope is used.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is a schematic view illustrating an example of a distance measuring unit in which an interferometer is configured with reference light and return light (reflection light) from an object to be measured using a pulsed laser.

FIG. 2 is a schematic view illustrating the pulsed laser emitted from a light source.

FIG. 3 is a schematic view illustrating an input/output characteristic of an SOA.

FIG. 4A is a schematic view illustrating how an output characteristic of a light-receiving element changes in accordance with a temporal difference between the reference light and the reflection light.

FIG. 4B is a schematic view illustrating how an output characteristic of a light-receiving element changes in accordance with a temporal difference between the reference light and the reflection light.

FIG. 4C is a schematic view illustrating how an output characteristic of a light-receiving element changes in accordance with a temporal difference between the reference light and the reflection light.

FIG. 5 is a characteristic diagram illustrating a relationship between a mirror position x and an output of the light-receiving element.

FIG. 6 is a schematic view illustrating an example of a system applied to a distance measurement of an image observed by an endoscope.

FIG. 7 is a schematic view illustrating a configuration of a signal processing block.

FIG. 8 is a schematic view illustrating an example in which the distance measuring unit is constituted by an optical fiber optical system.

FIG. 9 is a schematic view illustrating an example in which light is received by using PMT, HPD, or APD, instead of using the SOA, in the configuration in FIG. 8.

FIG. 10 is a schematic view illustrating an example in which a light-receiving signal is deteriorated in the light-receiving element.

FIG. 11 is a schematic view illustrating an example of the image observed by the endoscope.

FIG. 12 is a schematic view illustrating an example in which reference laser light is introduced at a stage prior to a scan unit.

FIG. 13 is a schematic view for explaining how an XYZ position is calculated.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Note that description will be provided in the following order.

1. Distance measuring unit according to present embodiment

2. Configuration examples of distance measuring system

3. Configurations of signal processing block

4. Specific configuration examples of distance measuring unit

5. Processing for measuring distance

1. Distance Measuring Unit According to Present Embodiment

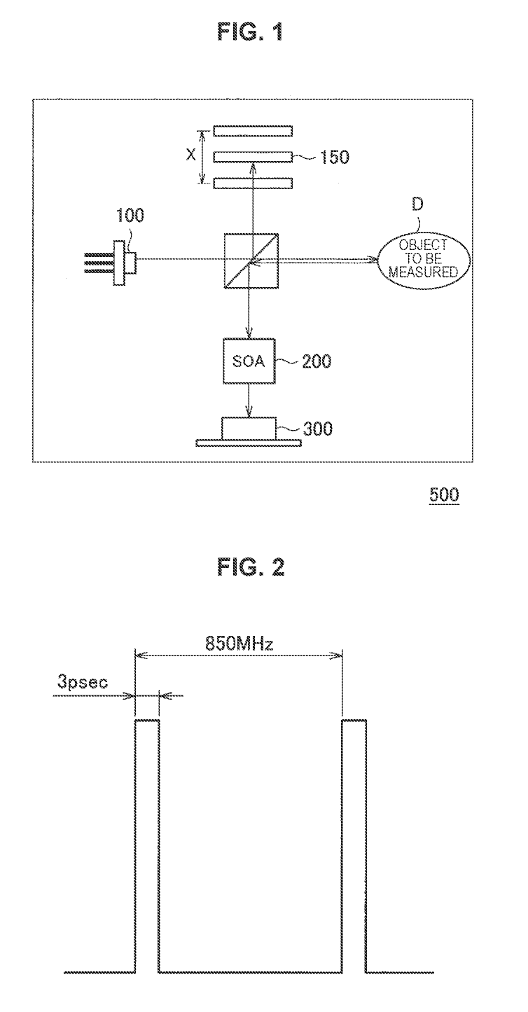

FIG. 1 is a schematic view illustrating an example of a distance measuring unit 500 in which an interferometer is configured with reference light and return light (reflection light) from an object to be measured using a pulsed laser. In this configuration, light obtained by interference between the reference light that is radiated from a light source 100 and reflected by a mirror 150 and the reflection light from an object to be measured is caused to transmit an SOA (Semiconductor Optical Amplifier) 200 and received by a light-receiving element (a photodetector: PD) 300. In this configuration, the SOA 200 is also used in an optical communication industry and has frequency characteristics capable of sufficiently coping with a communication wavelength signal band of several tens of GHz or more.

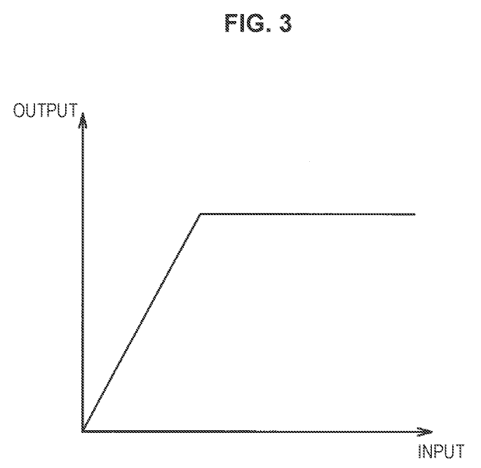

In the present embodiment, the distance measurement is performed by radiating a pulsed laser to an object to be measured similarly to the technology described above. FIG. 2 is a schematic view illustrating the pulsed laser emitted from the light source 100. This pulsed laser is emitted from the light source 100, which has, for example, an MOPA (Master Oscillator Power Amplifier) structure and emits pulsed laser light of 2 [psec] with a repetition frequency of 850 [MHz]. The light source 100 having the repetition frequency of 850 [MHz] emits a pulsed laser every 1.17 [nsec], thus pulsed light propagates in space at intervals of 35 [cm] in the air and about 26 [cm] in water by a distance.

In FIG. 2, only one pulse is preferably emitted in a measuring range of 0 to 40 [mm], and a pulse interval in water is preferably 80 [mm] or longer. Thus, the pulse repetition frequency is preferably set to 2.8 [GHz] or less.

Further, a detection resolution of 0.1 [mm] is converted to 0.5 [psec] in terms of time. In the case of using a method in which a center is estimated from shapes on both sides, the center can be estimated in a range of about 2 [mm]. Thus, a condition of a pulse width corresponding to 2 [mm] is preferably set to about 10 [psec] or less.

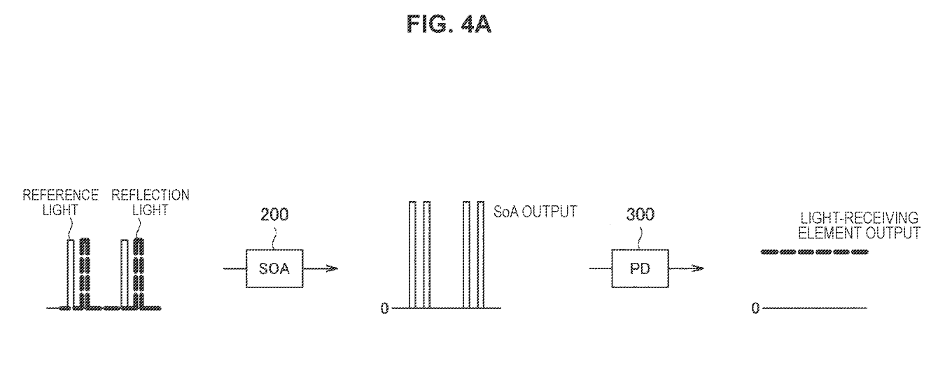

FIG. 3 is a schematic view illustrating an input/output characteristic of the SOA 200. As shown in FIG. 3, the SOA 200 through which the light obtained by interference between the reference light and the reflection light from an object to be measured is caused to pass has characteristics such that an output signal of the SOA 200 is limited by an element, specifically, the SOA 200 has characteristics such that an output of the SOA 200 is saturated in the case where an input signal of a certain level of power or more is inputted. In the present embodiment, a distance to an object to be measured is measured using such characteristics of the SOA 200.

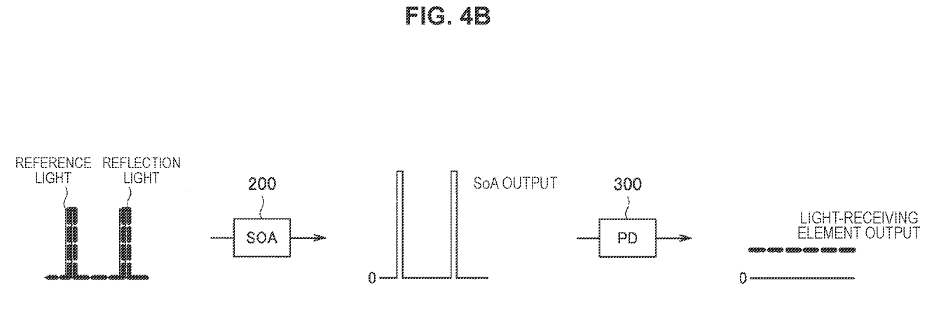

Next is an explanation of how an output characteristic of a light-receiving element 300 changes in accordance with a temporal difference between the reference light and the reflection light by referring to FIG. 4A to FIG. 4C. As shown in FIG. 4A, in the case where a pulse of the reference light and a pulse of the reflection light from a specimen (an object to be measured) are inputted into the SOA 200 in a temporally non-overlapping manner, each of the reference light and the reflection light is sufficiently amplified by the SOA 200 and outputted. On the other hand, as shown in FIG. 4B, in the case where 2 pulses are inputted into the SOA 200 in a temporally overlapping manner by adjusting a position of a mirror 150 (a distance x in FIG. 1), the output of the SOA 200 is saturated without being sufficiently amplified. As shown in FIG. 4C, in the case where the position of the mirror 150 is adjusted again such that the pulse of the reference light and the pulse of the reflection light from the specimen are temporally shifted, each of the reference light and the reflection light is sufficiently amplified by the SOA 200.

In such a configuration, if the output signal from the SOA 200 is received by the light-receiving element having a response frequency of, for example, about 10 [MHz], it is difficult to measure an individual light pulse emitted at a frequency of 850 [MHz] as shown in FIG. 2, thus the measurement is made for average energy of the individual light pulses. Consequently, as compared to the case where the pulse of the reference light and the pulse of the reflection light from the specimen are temporally shifted (see FIGS. 4A and 4C), in the case where the pulses of the reference light and the reflection light are temporally overlapping (see FIG. 4B), the amplification by the SOA 200 is not sufficiently achieved, thereby making the output signal detected by the light-receiving element 300 smaller.

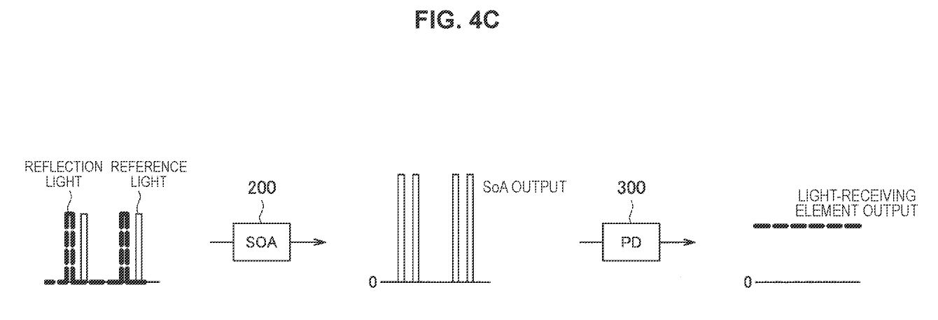

FIG. 5 is a characteristic diagram illustrating a relationship between a position x of the mirror 150 and an output of the light-receiving element 300. As shown in FIG. 5, when two pulses of the reference light and the reflection light are temporally overlapping, the amplification by the SOA 200 is not sufficiently achieved, thereby making the output of the light-receiving element 300 smaller as compared to the case where two pulses are not temporally overlapping. Thus, according to the present embodiment, it is possible to obtain information regarding whether two pulses are overlapping from the output signal of the light-receiving element 300 even if a time response band is insufficient.

2. Configuration Examples of Distance Measuring System

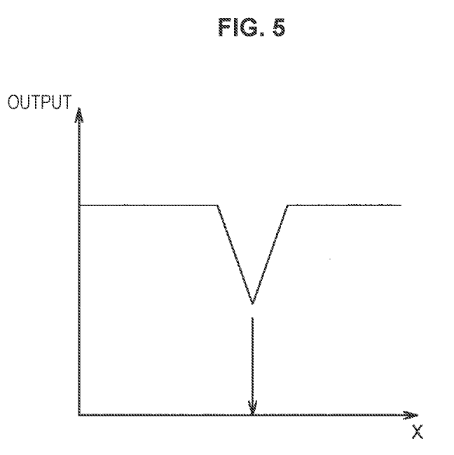

FIG. 6 is a schematic view illustrating an example of a system 1000 in which the above-mentioned principle is applied to the distance measurement of an image observed by an endoscope. This system 1000 includes the distance measuring unit 500 shown in FIG. 1, a scan unit 600, and an endoscope (an arthroscope) 700. As the endoscope 700, a plurality of types of endoscopes having different optical systems can be installed in the scan unit 600. The endoscope includes an imaging element 705, a mirror 720, and a lens 730.

This system 1000 measures a distance L to an object D to be measured by radiating a pulsed laser focused in a spot shape from the light source 100 to the object to be measured while being observed by the endoscope 700 through a control of galvano mirrors 610 and 620 of the scan unit 600, and obtaining a time required for return light to return from a position of the object. The distance measuring unit 500 and the scan unit 600 are connected via an optical fiber. A combined distance of the endoscope 700 and the optical fiber is generally longer than 35 [cm] corresponding to a space interval of the pulsed light having a frequency of 850 [MHz]. Thus, when the system is used in combination with the endoscope 700, a mirror is arranged at a position of a specimen-side end surface 710 of the endoscope 700 to perform a calibration once at this position. The position x of the mirror 150 on a reference light side is adjusted at the time of calibration, so that a moving distance of the mirror 150 from a position at the time of calibration corresponds to the distance L from the end surface 710 of the endoscope 700 to the object D to be measured.

In other words, the configuration is performed such that temporal phases of the reference light and the reflection light coincide with each other at the time of calibration. If a distance from the end surface 710 to the object D to be measured is L at the time of measurement, an optical path of the reflection light increases by 2×L. Then, a moving amount x of the mirror 150 is obtained by moving the mirror 150 from the position at the time of calibration to a position where the output of the SOA 200 is saturated, that is, a position where the output of the light-receiving element 300 shown in FIG. 5 decreases. This moving amount x of the mirror 150 corresponds to the increase of the optical path of the reflection light, thus making it possible to obtain the distance from the end surface 710 to the object D to be measured on the basis of the moving amount x.

In FIG. 6, as the distance L, a desired measuring range is preferably set to about 0 to 40 [mm]. This is because a size of a meniscus of the knee is about 30 to 35 [mm]. Further, a measurement resolution Ad is preferably about 0.1 to 1 [mm]. This is because a maximum resolution of currently commercially available MRI is about 80 um and a resolution of 100 μm or more is not demanded.

3. Configurations of Signal Processing Block

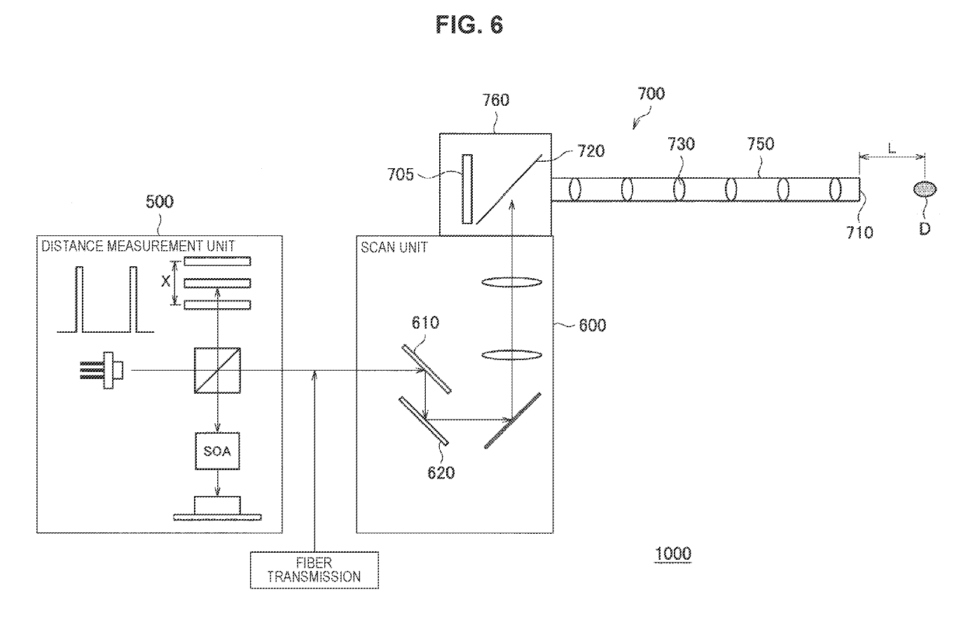

FIG. 7 is a schematic view illustrating a configuration of a signal processing block. As shown in FIG. 7, the signal processing block includes a distance measuring engine 500, a CCU 800, a scan mirror control unit 900, and a PC 950.

The CCU 800, which is a unit for mainly controlling the endoscope 700, acquires image data obtained by imaging of the imaging element 705. The image data acquired by the CCU 800 are sent to the PC 950 and the distance measuring unit 500.

The scan mirror control unit 900 receives information regarding an object area of which distance measurement data is obtained from the distance measuring unit 500 and sends a control signal for controlling the galvano mirrors 610 and 620 to the scan unit 600 on the basis of this information, thereby controlling the galvano mirrors 610 and 620. This operation allows the laser light emitted from the light source 100 to radiate to the object area.

The PC 950 sends the information regarding the object area of which the distance measurement data is obtained to the distance measuring unit 500. The distance measuring unit 500 makes the distance measurement in the object area and sends the distance measurement data obtained from the output signal of the light-receiving element 300 to the PC 950. In this configuration, the PC 950 has no need to include a keyboard, a display, and the like, as long as it has a function of performing a necessary arithmetic operation.

The distance measuring unit 500 includes a distance measuring portion 510 that obtains the distance to the object D to be measured from a relationship between the position x of the mirror 150 and light-receiving characteristics of the light receiving element 200 for a saturated light quantity outputted from the SOA 200. Note that the distance measuring portion 500 may be included in the PC 950.

4. Specific Configuration Examples of Distance Measuring Unit

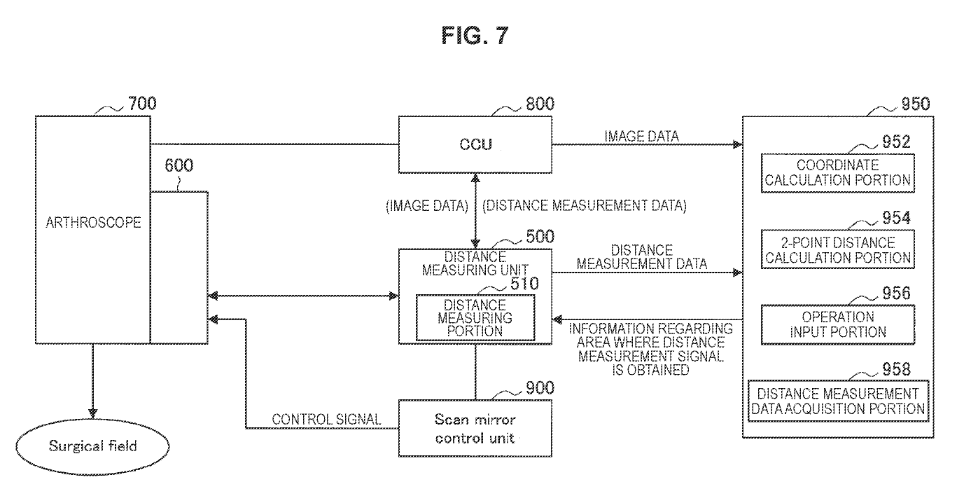

FIG. 8 is a schematic view illustrating an example in which the distance measuring unit 500 is constituted using an optical fiber optical system. This optical system can be constituted with optical fiber components (1×2 couplers 400, 410, and 420) as shown in the figure. Such an optical system can be constituted without requiring an optical surface plate and the like, and is thus robust and excellent in vibration resistance characteristics. Note that a VOA 430 is provided for adjusting a light intensity.

Further, as shown in FIG. 8, in the case where a polarization direction of the reflection light is changed by a structure or the like of the optical system on the midway or the object to be observed, a depolarizer 440 or the like for randomizing the polarization direction of the reflection light can be inserted to prevent a change in the quality of the detection signal.

Note that a signal light-receiving portion in which light amplified by the SOA 200 is received by the light-receiving element 300 may be, for example, a device having characteristics such that an output is saturated with a large instantaneous signal, such as a Geiger counter. As an example of the device in which the output is saturated with a first large instantaneous signal, a PMT or the like can be mentioned. However, the PMT includes a plurality of amplification means, thus time information becomes obscured as a transition is made from an initial amplification stage to a later amplification stage after several stages. In the present embodiment, even if two pulses of the reference light and the reflection light are slightly shifted to each other, these two pulses overlap in a temporal manner in a later amplification stage. Thus, it is anticipated that detection accuracy decreases as compared to the case of using the SOA 200 having frequency characteristics sufficient for accurate signal detection.

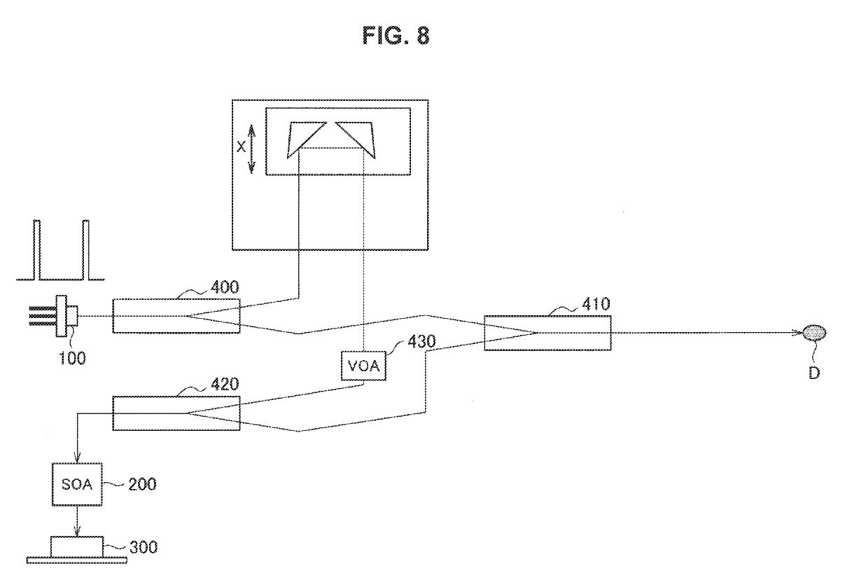

Note that, since this signal deterioration is caused by a large number of the amplification stages in the PMT, the signal deterioration can be reduced by using an HPD (Hybrid Photo Detector) or the like having less number of the amplification stages than the PMT. Further, if the light quantity of the return light is relatively sufficient, an APD (Avalanche Photo Diode) can also be used. FIG. 9 is a schematic view illustrating an example in which light is received by using the PMT, the HPD, or the APD, instead of using the SOA 200, in the configuration in FIG. 8.

Note that, in the signal light-receiving system described above, signal sensitivity increases most in the case where intensities of the reference light and the reflection light from the specimen are substantially equal to each other, thus as shown in FIG. 8 and FIG. 9, a VOA (Variable Optical Attenuator) 430 for adjusting an output can be arranged in the optical path of the reference light. In such a configuration, the light quantities of the reflection light from the specimen and the reference light can be made substantially equal, thereby enabling to increase the quality of signal.

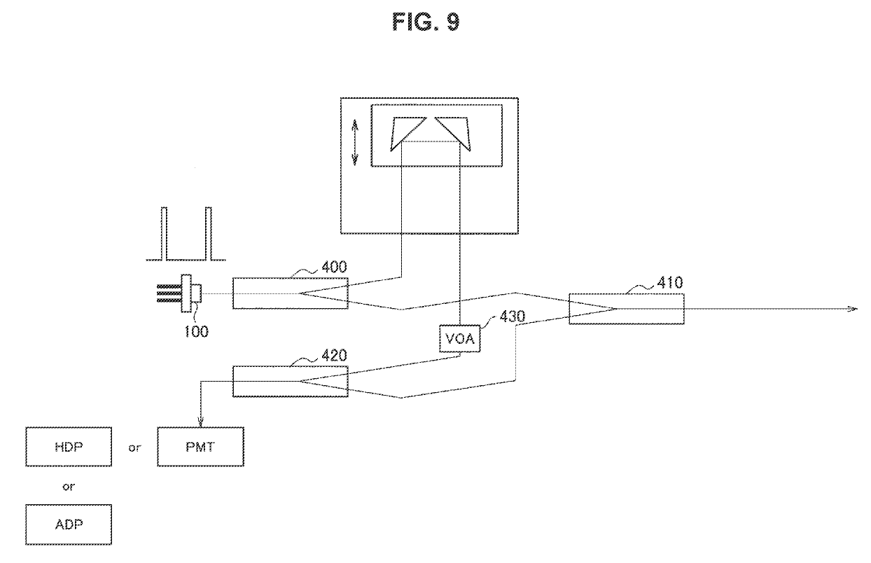

Further, in the case where a light reception signal in the light-receiving element 300 is deteriorated and results in a signal shown in FIG. 10, rather than obtaining a signal shown in FIG. 5, the following method is used. Intersections P1 and P2 between a certain threshold h and the light reception signal are obtained and the position x is calculated by determining a middle position P3 between P1 and P2.

A wavelength of the reference light used in the present embodiment is not particularly limited, however, in the case where a measurement is made in an underwater environment, loss of the reflection light from the specimen can be reduced by using light having a wavelength that attenuates less when propagating in water (e.g., 405 [nm]).

5. Processing of Measuring Distance

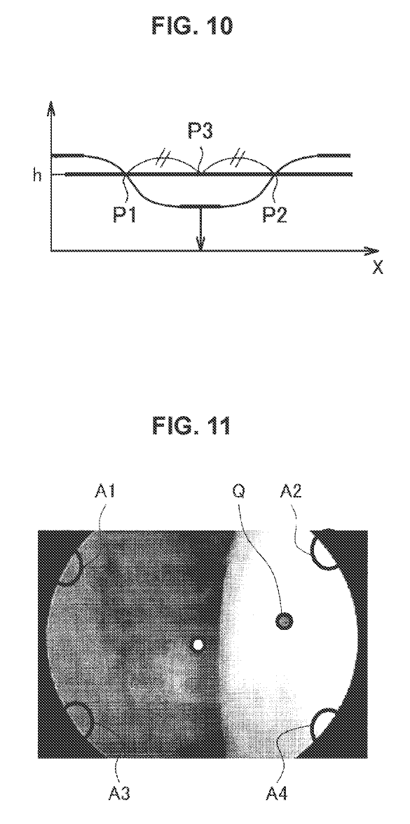

Next, an explanation is given to actual processing of the distance measurement of an observation image in the system in FIG. 6. FIG. 11 is a schematic view illustrating an example of an image observed by the endoscope 700 and shows an internal tissue of a body. The image shown in FIG. 11 is captured by the imaging element 705 of the endoscopes 700.

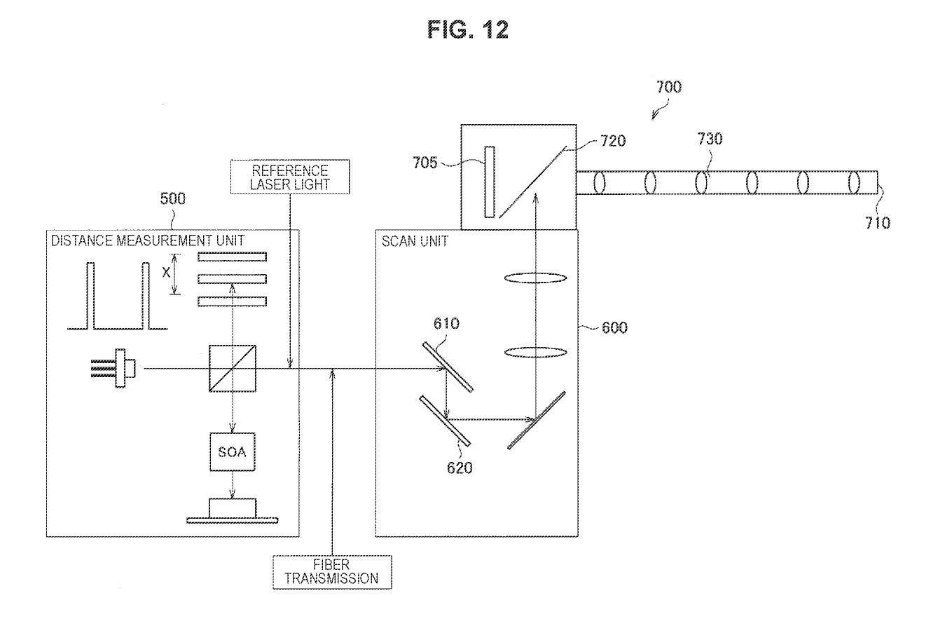

As shown in FIG. 11, an irradiation position Q is irradiated with laser light emitted from the light source 100. The laser light emitted from the light source 100 is not visible light, thus reference laser light is introduced at a stage prior to the scan unit 600 as shown in FIG. 12.

The reference laser light is a visible light laser beam that is introduced into the scan unit 600 from a light source different from the light source 100. The reference laser beam is superimposed on the laser light from the light source 100 and radiated to the object D to be measured via the scan unit 600 and the endoscopes 700. This causes a mark made by the reference laser light to appear at the irradiation position Q shown in FIG. 11.

The reference laser light can be placed on a location where the measurement is desired to be made by a measuring person by controlling the galvano mirrors 610 and 620. The measuring person initiates the distance measurement after confirming from the image that the irradiation position Q indicated by the reference laser beam reaches a desired measurement location. The reference laser beam is preferably switched off during the distance measurement, or the reference laser beam may be blocked by a wavelength filter or the like not to enter the SOA 200.

As described above, the position x of the mirror 150 on the reference light side is adjusted at the time of calibration, so that the moving distance of the mirror 150 from the position at the time of calibration corresponds to the distance L from the end surface of the endoscope 700 to the irradiation position Q.

In accordance with the procedures described above, it is possible to obtain distance information regarding a site desired to be measured (the irradiation position Q). In the procedures, in the case where the optical system of the endoscope 700 is a fisheye lens, position correction is performed by correcting curvature aberration or the like using design data of the optical system or measured data of the optical system, and an XYZ position (a coordinate) in a space is calculated from an XY position on the image and a measured optical propagation time from the end surface of the endoscope 00.

In the case of the arthroscope, a lens 730 is arranged symmetrically about an optical axis. That is, a cylindrical lens is not included. Further, a center (an optical axis C) of a barrel of the arthroscope forms a straight line. Further, information regarding mechanical variation in connecting a barrel 750 with a camera 760 of the arthroscope can be obtained from a camera image.

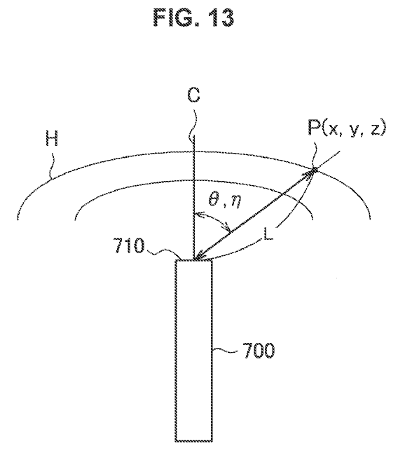

FIG. 13 is a schematic view for explaining how the XYZ position is calculated. In FIG. 13, a plane H, which is originally perpendicular to the optical axis C, is deformed into a curved shape by the curvature aberration of the lens 730. As shown in FIG. 13, a pixel position of a specified location on the image defines angles θ and η (θ and η represent angles in a horizontal and vertical directions, respectively) formed by the optical axis C of the endoscope 700 and an observation point (the irradiation position Q) on the observation image. Further, the distance (time) L to the measurement place is obtained by the distance measurement using the light source 100. Thus, XYZ coordinates of the observation point (the irradiation position Q) can be obtained by performing a conversion using a conversion table. In the calculation, pixel data (pixel numbers) on the image and the above-mentioned angles θ and η formed by the optical axis differ depending on the respective endoscopes (the optical systems), thus calculation of the conversion can be easily performed by preparing in advance a matrix of conversion data for each endoscope. To determine a distance between two points specified on the image, the XYZ coordinates of these two points are obtained and then the difference of their XYZ coordinates is calculated.

Specifically, the XYZ coordinates of an arbitrary point on the image can be calculated by the following processings from step 0 to step 4.

### (Step 0)

Coordinates of the optical axis C of the endoscope 700 on the image are obtained. In this example, the origin XY coordinates (X0, Y0) representing a center of the barrel of the arthroscope 700 are obtained from boundaries of the image (contours of the visual field) in regions A1 to A4 in FIG. 11. This process can correct a mechanical installation error. Note that Z represents a distance in the optical axial direction (in a depth direction of the image), X represents a distance from the origin in a horizontal direction of the image, where a left side is +, and Y represents a distance from the origin in a vertical direction of the image, where an upside is +. A reference position Z0 in the Z direction is the end surface 710 at a tip of the endoscope 700.

### (Step 1)

Coordinates (X1, Y1) of the observation point (the irradiation position Q) where the distance measurement is desired to be made are obtained.

### (Step 2)

A distance P on the image data between the coordinates (X1, Y1) of the observation point (the irradiation position Q) and the origin coordinates (X0, Y0) is calculated by the following formula.

P=((X1−X0)²+(Y1−Y0)²)^{0.5}

### (Step 3)

The angle θ of the observation point (the irradiation position Q) from the optical axis C is obtained from a conversion table using the measured distance L from the end surface 710 to the object D to be measured and the calculated distance P. An example of the conversion table is shown below. Note that the angle θ can be obtained by applying the distance P into a vertical axis of the conversion table and applying the distance L into a lateral axis of the conversion table.

TABLE 1

| | | Distance (L) | | | | | |
|---|---|---|---|---|---|---|---|
| | | 0 | 1 | 2 | 3 | 4 | 5 |
| delta P | 0 | 0 | 0 | 0 | 0 | 0 | 0 |
| | 50 | 5.720202 | 5.748803 | 5.777547 | 5.806435 | 5.835467 | 5.864644 |
| | 100 | 11.38325 | 11.44017 | 11.49737 | 11.55485 | 11.61263 | 11.67069 |
| | 150 | 16.93256 | 17.01722 | 17.10231 | 17.18782 | 17.27376 | 17.36013 |
| | 200 | 22.31269 | 22.42425 | 22.53637 | 22.64905 | 22.7623 | 22.87611 |
| | 250 | 27.46987 | 27.60722 | 27.74526 | 27.88398 | 28.0234 | 28.16352 |
| | 300 | 32.35258 | 32.51435 | 32.67692 | 32.8403 | 33.00451 | 33.16953 |
| | 350 | 36.91204 | 37.0966 | 37.28209 | 37.4685 | 37.65584 | 37.84412 |
| | 400 | 41.10269 | 41.3082 | 41.51474 | 41.72232 | 41.93093 | 42.14058 |

### (Step 4)

The XYZ coordinates are obtained using L and θ.

X=L·sin θ·cos η, Y=L·sin θ·sin η, Z=L·cos θ

Once the XYZ coordinates of the arbitrary point on the image are obtained as described above, the distance between two points on the image can be calculated from the XYZ coordinates of the respective points.

The calculation of the XYZ coordinates and the arithmetic operation of the distance between two points described above are respectively performed by a coordinate calculation portion 952 and a 2-point distance calculation portion 954 in the PC 950. Further, an operation input portion 956 in the PC 950 acquires information regarding an object area where the distance measurement data are obtained (the coordinates (X1, Y1) of the irradiation position Q) by an operation input from a user. Further, a distance measurement data acquisition portion 958 in the PC 950 acquires distance measurement data L from the distance measuring unit 500. The coordinate calculation portion 952 acquires the origin XY coordinates (X0, Y0) from the image data obtained by imaging of the imaging element 705 and calculates the XYZ coordinates of the observation point (the irradiation position Q) in accordance with (step 1) to (step 4). Further, the 2-point distance calculation portion 954 calculates a distance between arbitrary 2 points on the basis of the XYZ coordinates of these 2 points. Note that the coordinate calculation portion 952 and the 2-point distance calculation portion 954 may be provided on a side of the distance measuring unit 500.

As described above, according to the present embodiment, it becomes possible to make an accurate distance measurement in an optical system environment with strong curvature aberration, such as a fisheye lens, for example, in an a small-diameter endoscopic observation environment, to which it is difficult to apply a usual distance measurement method, such as a stereoscopic measurement method.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

A distance measurement device including:

a light source configured to emit pulsed laser light;

a superimposition portion configured to superimpose reflection light obtained by reflection of the pulsed laser light by an object to be measured and reference light that is the pulsed laser light;

a saturation output portion, on which the reference light and the reflection light superimposed on each other are made incident, configured to output light having a saturated light quantity when incident light reaches a predetermined light quantity due to superimposition of pulses of the reflection light and the reference light; and a light-receiving portion configured to receive the light outputted from the saturation output portion.

(2)

The distance measurement device according to (1), in which

the superimposition portion includes a superimposition mirror configured to reflect the reference light superimpose the reference light on the reflection light, and

the distance measurement device includes a distance measuring portion configured to obtain a distance to the object to be measured from a relationship between a position of the superimposition mirror and an output of the light having the saturated light quantity.

(3)

The distance measurement device according to (2), in which

the pulsed laser light is radiated from an endoscope to the object to be measured and the reflection light from the object to be measured is superimposed on the reference light,

the superimposition mirror is arranged at a first position where the output of the saturation output portion is saturated at a time of calibration during which the pulsed laser light is reflected at a tip position of the endoscope,

the superimposition mirror is arranged at a second position where the output of the saturation output portion is saturated at a time of measurement during which the pulsed laser light is reflected by the object to be measured, and

the distance measuring portion obtains the distance to the object to be measured on a basis of the first position and the second position.

(4)

The distance measurement device according to any of (1) to (3), in which the light source includes an MOPA and has a pulse repetition frequency of 2.8 GHz or less.

(5)

The distance measurement device according to any of (1) to (4), in which the saturation output portion includes an SOA.

(6)

An imaging system including:

a distance measuring unit including a light source configured to emit pulsed laser light, a superimposition portion configured to superimpose reflection light obtained by reflection of the pulsed laser light by an object to be measured and reference light that is the pulsed laser light, a saturation output portion, on which the reference light and the reflection light superimposed on each other are made incident, configured to output light having a saturated light quantity when incident light reaches a predetermined light quantity due to superimposition of pulses of the reflection light and the reference light, and a light-receiving portion configured to receive the light outputted from the saturation output portion; and

an endoscope unit including an endoscope configured to emit the pulsed laser light made incident in the endoscope to the object to be measured, an imaging element configured to image the object to be measured as an object by the endoscope, and an adjustment portion configured to adjust a direction of the pulsed laser light such that the pulsed laser light is radiated to a specified location of the object to be measured.

(7)

The imaging system according to (6), in which

the superimposition portion includes a superimposition mirror for reflecting the reference light to superimpose the reference light on the reflection light, and

the imaging system includes a distance measuring portion configured to obtain a distance to the object to be measured from a relationship between a position of the superimposition mirror and an output of the light having the saturated light quantity.

(8)

The imaging system according to (7), in which

the pulsed laser light is radiated from the endoscope to the object to be measured and the reflection light from the object to be measured is superimposed on the reference light,

the superimposition mirror is arranged at a first position where the output of the saturation output portion is saturated at a time of calibration during which the pulsed laser light is reflected at a tip position of the endoscope,

the superimposition mirror is arranged at a second position where the output of the saturation output portion is saturated at a time of measurement during which the pulsed laser light is reflected by the object to be measured, and

the distance measuring portion obtains the distance to the object to be measured on a basis of the first position and the second position.

(9)

The imaging system according to (7), including:

a correction portion configured to correct the distance to the object to be measured on a basis of the distance to the object to be measured obtained by the distance measuring portion, the specified location on an image captured by the imaging element, and an optical characteristic of curvature aberration in imaging of the imaging element.

(10)

The imaging system according to any of (6) to (9), in which the light source includes an MOPA and has a pulse repetition frequency of 2.8 GHz or less.

(11)

The imaging system according to any of (6) to (10), in which the saturation output portion includes an SOA.

### REFERENCE SIGNS LIST

100 light source
150 mirror
200 SOA
300 light-receiving element
500 distance measuring unit
1000 system

1. A distance measurement device comprising:
a light source configured to emit pulsed laser light;
a superimposition portion configured to superimpose reflection light obtained by reflection of the pulsed laser light by an object to be measured and reference light that is the pulsed laser light;
a saturation output portion, on which the reference light and the reflection light superimposed on each other are made incident, configured to output light having a saturated light quantity when incident light reaches a predetermined light quantity due to superimposition of pulses of the reflection light and the reference light; and
a light-receiving portion configured to receive the light outputted from the saturation output portion.

2. The distance measurement device according to claim 1, wherein
the superimposition portion includes a superimposition mirror configured to reflect the reference light superimpose the reference light on the reflection light, and
the distance measurement device includes a distance measuring portion configured to obtain a distance to the object to be measured from a relationship between a position of the superimposition mirror and an output of the light having the saturated light quantity.

3. The distance measurement device according to claim 2, wherein
the pulsed laser light is radiated from an endoscope to the object to be measured and the reflection light from the object to be measured is superimposed on the reference light,
the superimposition mirror is arranged at a first position where the output of the saturation output portion is saturated at a time of calibration during which the pulsed laser light is reflected at a tip position of the endoscope,
the superimposition mirror is arranged at a second position where the output of the saturation output portion is saturated at a time of measurement during which the pulsed laser light is reflected by the object to be measured, and
the distance measuring portion obtains the distance to the object to be measured on a basis of the first position and the second position.

4. The distance measurement device according to claim 1, wherein the light source includes an MOPA and has a pulse repetition frequency of 2.8 GHz or less.

5. The distance measurement device according to claim 1, wherein the saturation output portion includes an SOA.

6. An imaging system comprising:
a distance measuring unit including a light source configured to emit pulsed laser light, a superimposition portion configured to superimpose reflection light obtained by reflection of the pulsed laser light by an object to be measured and reference light that is the pulsed laser light, a saturation output portion, on which the reference light and the reflection light superimposed on each other are made incident, configured to output light having a saturated light quantity when incident light reaches a predetermined light quantity due to superimposition of pulses of the reflection light and the reference light, and a light-receiving portion configured to receive the light outputted from the saturation output portion; and
an endoscope unit including an endoscope configured to emit the pulsed laser light made incident in the endoscope to the object to be measured, an imaging element configured to image the object to be measured as an object by the endoscope, and an adjustment portion configured to adjust a direction of the pulsed laser light such that the pulsed laser light is radiated to a specified location of the object to be measured.

7. The imaging system according to claim 6, wherein
the superimposition portion includes a superimposition mirror for reflecting the reference light to superimpose the reference light on the reflection light, and
the imaging system includes a distance measuring portion configured to obtain a distance to the object to be measured from a relationship between a position of the superimposition mirror and an output of the light having the saturated light quantity.

8. The imaging system according to claim 7, wherein
the pulsed laser light is radiated from the endoscope to the object to be measured and the reflection light from the object to be measured is superimposed on the reference light,
the superimposition mirror is arranged at a first position where the output of the saturation output portion is saturated at a time of calibration during which the pulsed laser light is reflected at a tip position of the endoscope,
the superimposition mirror is arranged at a second position where the output of the saturation output portion is saturated at a time of measurement during which the pulsed laser light is reflected by the object to be measured, and
the distance measuring portion obtains the distance to the object to be measured on a basis of the first position and the second position.

9. The imaging system according to claim 7, comprising:
a correction portion configured to correct the distance to the object to be measured on a basis of the distance to the object to be measured obtained by the distance measuring portion, the specified location on an image captured by the imaging element, and an optical characteristic of curvature aberration in imaging of the imaging element.

10. The imaging system according to claim 6, wherein the light source includes an MOPA and has a pulse repetition frequency of 2.8 GHz or less.

11. The imaging system according to claim 6, wherein the saturation output portion includes an SOA.
